(19) United States
(12) Patent Application Publication
(10) Pub. No.: US 2005/0260610 A1
(43) Pub. Date: Nov. 24, 2005

(54) Method for diagnosing and prescribing a regimen of therapy for human health risk

(76) Inventors: Richard E. Kurtz, Berwyn, PA (US); Ronald B. David, Mechanicsville, VA (US)

Correspondence Address: Richard E. Kurtz, Esquire, 380 Keller Road, Berwyn, PA, 19312, US

(21) Appl. No.: 10/909,032

(22) Filed: Jul. 30, 2004

Related U.S. Application Data
(63) Continuation-in-part of application No. 10/849,755, filed on May 20, 2004, now abandoned.

(51) Int. Cl. G06F 17/60; C12Q 1/00; C12Q 1/68; G06F 19/00; G01N 33/48; G01N 33/50

(52) U.S. Cl. 435/6; 702/20; 705/3

(57) ABSTRACT

A patient is diagnosed and a regimen of treatment is prescribed in a system which tests the patients, receives test results from a patient, including DNA test results. A database of human genome data correlating human health disorder with DNA genetic tests is accessed. A database of environmental factors which correlate known human health disorders with environmental conditions is accessed. Risk factors including genetic risk and environmental risk are developed, and from these, a composite risk factor is developed. This is used to access a database of prescribed drug therapy.

### CROSS-REFERENCE TO RELATED APPLICATION

This patent application is a continuation-in-part of U.S. patent application Ser. No. 10/849,755 filed May 20, 2004, the contents of which is hereby incorporated by reference.

### FIELD OF THE INVENTION

This invention relates to a computer system and method for diagnosing a patient and prescribing a regimen of treatment, and more particularly to testing the patient, receiving DNA data regarding the patient, receiving other relevant health information, receiving human genome data, receiving information regarding the correlation of environmental factors with health risks, and from the data received prescribing a regimen of drug therapy.

### BACKGROUND OF THE INVENTION

The total number of expressed genes or transcription units in a human genome is around 30,000-40,000 (Venter G. et al., Science 2000, Vol. 291: 1304-1351; The Genome International Sequencing Consortium, Nature 2000, Vol. 409: 860-921). Some researchers believe that up to 500,000 human RNA transcripts exist, and that more than 30% of genes or transcription units in the human genome produce several RNA splice variants. (Mironov et al. 1999, Genome Research 9: 1288-1293). This immense genomic data pool provides for a better understanding of human physiology and brings about significant therapeutic and diagnostic promises.

Individual genetic predisposition is a challenging area to biomedical researchers, genetic counselors, and clinicians. It is estimated that the DNA sequence of any two given human beings differ by a few percent. Different polymorphisms in the patient make the diagnosis and prescription for a patient difficult. These differences in DNA base composition can result in different protein functions, and thus become consequential with respect to the individual's physical well-being in some situations.

Lifestyle and dietary avoidance strategies are becoming feasible for a significant number of genetic diseases, which makes it extremely critical and beneficial to detect genetic predispositions early in time. That is, if an individual knows that a disease-responsible gene carries mutations in his or her genome, he or she may institute recommended changes in lifestyle and diet to postpone or avoid the outbreak of the disease. Therefore, the diagnostic and therapeutic promises of the human genome data may be realized as the data is transformed into personalized knowledge on individual genes and their impact on the onset and severity of a disease.

One example of patient testing is shown in PCT Patent Application No. WO 00/28460 (Maus et al.), assigned to Lifestream Technologies, Inc. This application discloses a health monitoring and diagnostic device with a network-based health assessment and medical records maintenance system

U.S. Patent Application 2003/0217037 A1 describes a secure medical records maintenance system that is specifically adapted for use with a health monitoring and diagnostic system. The maintenance system may store any type of electronic data, including a wide variety of medical records, for example electronic medical data generated remotely from the hospital or doctor's office environment.

Increasing attention and effort is being directed to providing large networks linking data bases of medical records. See for example, “Fix Of A Sick System”, Information Week, Dec. 15, 2003, which describes a data analysis project for the federal government centers for Medicare and Medicaid services. In this project, Premier Healthcare uses four Oracle9i databases to manage data relating to healthcare.

The federal government is spending over a billion dollars in 2004 on information technology (IT) including standardizing the format of patient data. See “No Slack in Government IT Demand” by Bill Snyder, TheStreet.com, Feb. 5, 2003.

It is an object of the present invention to provide a system which utilizes automated genetic testing, network technology and laboratory information system to implement an improved method and system for diagnosing and prescribing a regimen of therapy for human health risk.

### SUMMARY OF THE INVENTION

In accordance with the invention, a patient is diagnosed and a regimen of treatment is prescribed by a system which includes a personal computer utilized by the doctor and a remote computer which administers tests.

These tests are stored in a database which is analyzed against a genomic database of known health disorders.

In accordance with one aspect of the invention, the genomic database contains levels of risk factors. An environmental database also includes levels of risk factors associated with a patient's exposure to these environmental risks.

In a preferred embodiment, a composite risk factor is generated from the genetic risk factors and environmental risk factors. This composite risk factor is used to select a prescribed regimen of drug therapy from a drug database.

Further in accordance with the invention, a regimen of prescribed patient treatments may also be generated. These include suggested dietary constraints and activity level. The prescribed regimen of treatment is transmitted to the PC where it is utilized by the doctor.

Further in accordance with the invention, the patient tests are performed in test centers with Laboratory Information Systems having a common data format.

The foregoing and other objects, features, and advantages will be better understood from the following more detailed description and appended claims.

### BRIEF DESCRIPTION OF THE DRAWINGS

FIG. 1 depicts the computer system of the present invention; and

FIG. 2 is a flow chart depicting the operation of the invention.

### DETAILED DESCRIPTION OF ILLUSTRATIVE EMBODIMENTS

Computer System for Diagnosis and Prescribing

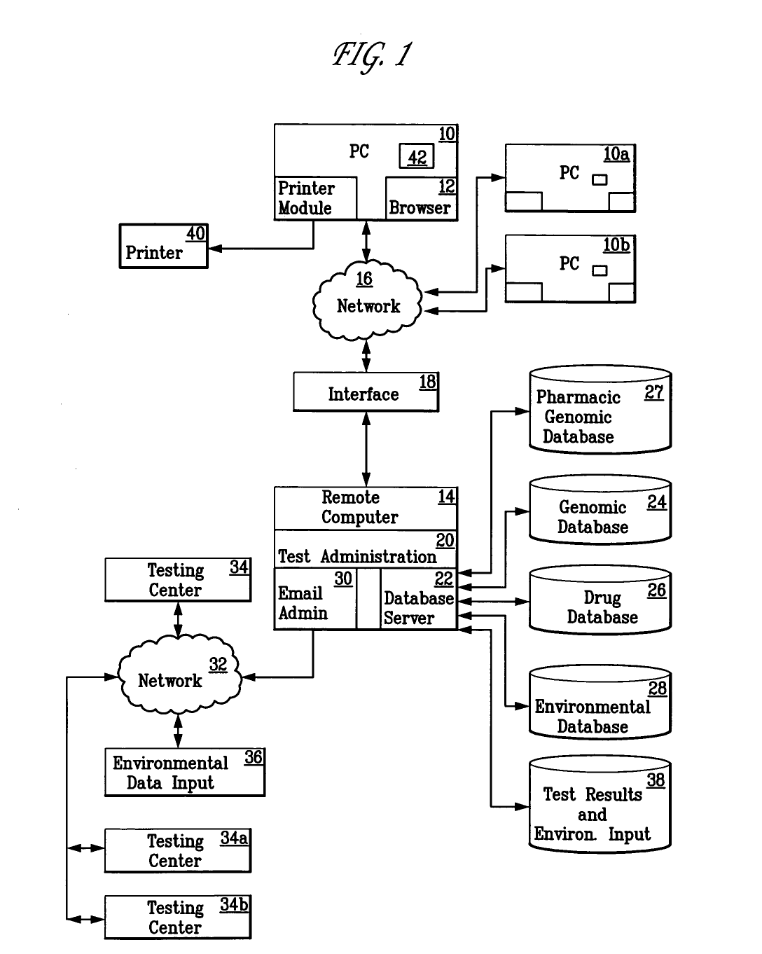

An example of a computer system suitable for use is depicted in FIG. 1.

A personal computer (PC) 10 at which a doctor can access the databases includes a browser 12. Personal computer 10 accesses a remote computer 14 through a network 16. Netscape Navigator or Microsoft Internet Explorer are common browsers. Network 16 is the internet, a local area network, or a virtual private network (VPN). A plurality of PC's including 10b and 10c access computer 14 through network 16.

Remote computer 14 runs a network interface program 18 such as Microsoft Interface Server or an Apache Server. Interface program 18 accepts instructions from the network 16. The instructions pass through to a test administration program 20 which is running on the remote computer 14. The interface program 18 uses the HTTPS protocol to transfer instructions from personal computer 10 to the remote computer 14.

Test administration program 20 includes a database server 22 which is connected to a genomic database 24, a prescribed drug database 26, a pharmacic genomic database 27, and an environmental database 28. The contents of these databases is described more fully below.

E-mail administration module 30 sends data related to a doctor's order through a network 32 to a testing center 34 and environmental data input 36.

Laboratory Information Systems (LIS) are known. See “LIS and the Enterprise” by Richard R. Rigoski. In accordance with the present invention, the LIS's use a standard format and are interconnected by the internet.

Testing and Environmental Input

A testing center 34, comprises a laboratory and a computer system. The testing center 34 performs the tests in an anonymous manner and returns the results of the tests over a network 32 to the database 38.

In the laboratory a blood sample is drawn and tested for the usual tests including a series of tests commonly referred to as Chem 20. The results of these tests are digitized. In addition to these tests, DNA in the blood sample is analyzed and put in a format which can be correlated with known genetic disorders.

Similarly, patient screening for environmental data is digitized at the data input 36. Typical patient screening includes predilection for smoking, exposure to asbestos, frequency of airline travel, caloric intake, weight to bone mass, and the like. These inputs are all transmitted over network 32 to the remote computer 14 and thence to the database 38.

Other testing centers including 34a and 34b in the system are connected to Remote Computer 14 over network 32 which is similar to, or the same as, network 16.

Genomic Database 24

The genomic database 24 provides access to the Celera Human Genome Sequence Data. In particular the database contains known correlations between genetic sequences and human health disorders. The GenBank or LIFESEQ databases are examples. The disorder correlations are expressed as a Health Risk Assessment One classification scheme based on genetics comprises a plurality of risk classes ranked from the lowest risk to the highest risk, relating to one or more disorders of interest. A personal health risk assessment with respect to one or more disorders determines a risk class based on test results.

For example, for a test that examines the sequences of one or more genes contributing to a disorder, a classification scheme may comprise the following risk classes:

(i) risk class I, no increased genetic risk of developing the disorder. This class represents the homozygous wild type group; both alleles of the gene are normal.

(ii) risk class II, usually moderately increased relative genetic risk of developing the disorder. This class represents the heterozygous wild type—mutant group; one allele of the gene is mutated.

(iii) risk class III, usually moderately to severely increased relative genetic risk of developing the disorder. This class represents the homozygous mutant group; both alleles of the gene is mutated.

(iv) risk class IV, usually highly increased relative genetic risk of developing the disorder. The class represents the combination of several mutations in different genes contributing to the disorder; different alleles of more than one gene are mutated. This typically is the worst class to be in.

Drug Database 26

The pharmacopeias of prescribed drugs for known disorders is large and growing. Known databases provide such information. Predisposition to adverse drug response is similarly known. See General of the America Association 79, 1200-1205 (1998).

Pharmacic Genomic Database 27

Dr. Francis Collins, Director of the National Human Genome Research Project, has identified the risk factor for major adverse reactions for certain drugs based on the genomics of the subject. In accordance with the present invention, the known risks are stored in Pharmacic Genomic Database 27. The classification is either risk or no risk of major adverse reactions for a given drug for a given genetic makeup, but more sophisticated classifications can be used.

For example, in treating a patient for allergies a profile of the patient's single nucleotide polymorphisms (SNP profile) is compared with the SNP profile of a subject known to have had an adverse reaction to a certain drug. If there is a match, the indication is Risk Class I.

Environmental Database 28

The environmental database 28 contains a classification of risk classes similar to that in genomic database 24.

For example, the classification scheme may comprise the following risk classes for developing a particular health disorder:
i) Risk Class I—No Increased Risk of Developing the Disorder;
ii) Risk Class II—Usually Moderately Increased Relative Risk of Developing the Disorder;
iii) Risk Class III—Usually Moderately to Severely Increased Relative Risk of Developing the Disorder;
iv) Risk Class IV—Usually Highly Increased Relative Risk of Developing the Disorder.

As an example, database server 22 would select Risk Class IV for Deep Vein Thrombosis from the Environmental Database if the environmental data input from the subject patient indicates frequent flying.

A risk class from the genomic date base 24 is further divided into subclasses to take into account, for example, the environmental or behavior factors, such as smoking, contraceptives, overweight, and immobilization.

Operation of the System

A doctor identifies a patient to be diagnosed on Personal Computer 10. Personal Computer 10 transmits a request for testing over network 16. This is analyzed by the test administration program 20 in remote computer 14 to determine a convenient testing center. Alternatively, the doctor designates the testing center 34. The prescription for testing is transmitted by hand or by e-mail administrator 30 and network 32 to the testing center 34. The appropriate tests are performed including a DNA analysis. The test results are transmitted over network 32 to the remote computer 14 where they are stored in database 38.

Environmental data regarding the patient including predilection to smoking, weight, dietary information and the like is collected as data input 36. Alternatively, this information may be collected by the doctor and inputted through PC 10. This information is also stored in the environmental database 38.

Remote computer 14 compares the DNA tests with risk factors for known genomic disorders in genomic database 24 to determine a risk factor of known health disorders for the subject patient. Similarly the environmental input from database 38 is compared with known environmental risks in environmental database 28. Computer 14 combines these two risk factors to develop a composite risk factor for a known disorder. This combined risk factor is used to search the drug database 26 to generate a prescribed drug therapy for the patient. The Pharmacic Genomics Database 27 is searched for risks of major adverse reactions. If none are identified remote computer 14 uses the composite risk factor to develop a regimen of treatment such as dietary and life-style changes. These are transmitted to personal computer 10 where they may be printed on printer 40 or displayed on monitor 42.

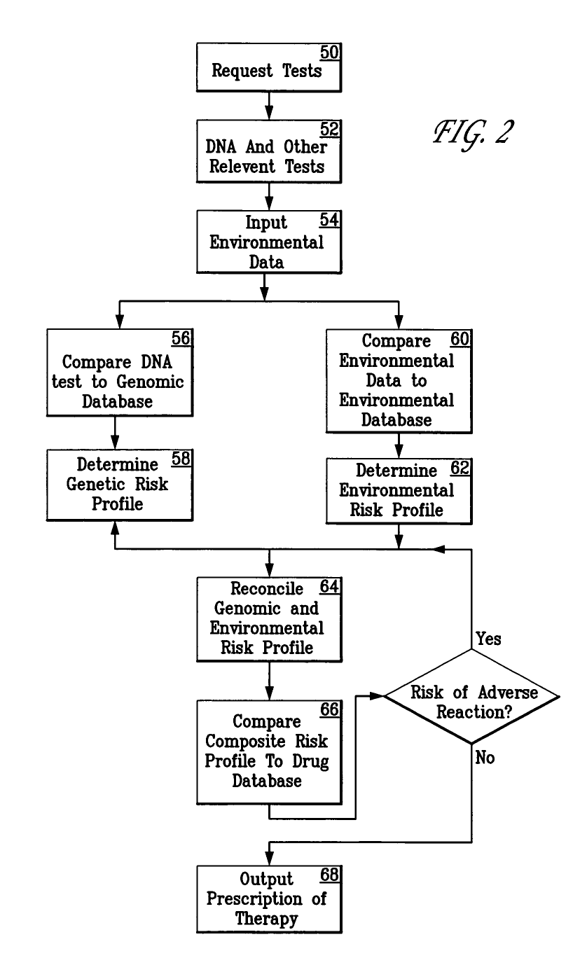

This is summarized in the flow sheet of FIG. 2. As indicated at 50, tests are requested by the doctor from the PC 10. These requests are transmitted to the testing center 34 where DNA and other relevant tests are performed as indicated by the step 52 in the flow sheet.

Environmental data are inputted as indicated by the step 54.

In step 55 DNA test results are compared to the genomic database. From this comparison 8, a genetic risk profile is developed at 51.

The environmental data of the patient is compared to the environmental database as indicated by the step 60. From this an environmental risk profile is developed as indicated at 62.

The genetic risk profile and the environmental risk profile are reconciled as indicted at 64. This develops a composite risk profile. This is compared to the drug database as indicated at 66. From this comparison a prescription of therapy is developed as indicated at 68.

### EXAMPLE 1

Genetic Risk Factors In Hemostasis And Deep Vein Thrombosis. This is a modification of Example 2 in U.S. Patent Application 2003/0217037 A1.

Certain individuals are genetically predisposed to develop deep venous thrombosis (DVT) which may lead to fatal lung embolism, especially when subject to immobilization during long air travel. The mortality rate caused by DVT is evidently higher than the mortality rate from aircraft crash. Recent studies indicate that there may be an increased frequency of DVT in the lower limb during long air travel; symptom-less DVT might occur in up to 10% of air travelers (The Lancet, 357, 1485-1489 (2001)).

The two most common genetic risk factors in patients with DVT is a single G-to-A base change at nucleotide 1691 (G1691A) in the factor V gene, termed factor V Leiden (FV-Leiden)-and a single G-to-A base change at nucleotide position 20210 (G20210A) within the 3′-unsaturated region of the prothrombine (PT) gene. The FV-Leiden mutation appears in 20-60% of patients with a known DVT history examined for a predisposition to DVT and occurs in approximately 5% of the western population.

Mutation screening therefore can classify long-haul airline travelers into two categories: those who are required to take precautions to prevent development of DVT (e.g., taking oral anticoagulants or wearing anti-thrombotic stockings) and those who are not subject to increased risks of DVT.

While a particular embodiment has been shown and described, various modifications may be made. All modifications within the true scope of the invention are covered by the appended claims.

1. A method of diagnosing a patient and prescribing a regimen of treatment on a computer system comprising:
testing said patient;
producing a test result from said patient including DNA test results and other relevant health test results;
accessing a database of human genome data correlating known human health disorders with DNA genetic tests;
accessing a database of environmental factors which correlate known human health disorders with environmental conditions;
accessing a database of prescribed drug therapy for human health disorders; and
prescribing a regimen of treatment based on access to said test results, access to said DNA database, access to said environmental database and access to said database of said prescribed drug therapy.

2. The method recited in claim 1 wherein the step of accessing a database of human genome data includes:
generating a genetic risk factor which correlates a known human health disorder with DNA sequences.

3. The method recited in claim 1 wherein the step of accessing a database of environmental factors includes generating an environmental risk factor which correlates a known human health disorder with a particular environmental factor.

4. The method recited in claim 1 wherein the step of accessing a database of human genome data includes:
generating a genetic risk factor which correlates a known human health disorder with DNA sequences; and
wherein the step of accessing a database of environmental factors includes generating an environmental risk factor which correlates a known human health disorder with a particular environmental factor.

5. The method recited in claim 4 further comprises:
generating a composite risk factor based on both said genetic risk factor and said environmental risk factor.

6. The method recited in claim 5 wherein the step of prescribing a regimen of treatment includes accessing a drug database and determining said prescribed drug therapy from said composite risk factor.

7. The method recited in claim 1 wherein the steps of testing and producing a test result are performed on Laboratory Information Systems operating on a common format.

8. The method recited in claim 1 further comprising:
accessing a pharmacic genomic database to determine if there is a risk for a major adverse reaction for a particular drug for said patient.

9. The method recited in claim 8 wherein said pharmacic genomic database contains a subject SNP profile of a subject known to have had an adverse reaction to a prescribed drug therapy and wherein said method further comprises:
testing said patient to obtain a patient's SNP profile;
comparing said patient's SNP profile to said subject SNP profile; and
indicating a Risk if there is a match in said comparing step.

10. A computer system for diagnosing a patient and prescribing a regimen of treatment comprising:
personal computers for use by doctors;
a remote computer having programs for test administration and database service;
a network interconnecting said personal computers with said remote computer;
a plurality of testing centers interconnected with said remote computer by a network; and
a plurality of databases having genomic, environmental and drug therapy information accessible by said remote computer;
said remote computer producing a diagnosis and a prescription of therapy for said patient from said information.

11. The system recited in claim 10 wherein said remote computer administers a test for DNA, produces a DNA test result, and compares said DNA test result with a database of known genetic disorders to develop a genetic risk factor for a known genetic disorder.

12. The system recited in claim 10 wherein said system receives environmental data for said patient and said remote computer compares said environmental data with a database of known health disorders correlated with environmental factors to generate an environmental risk factor.

13. The system recited in claim 12 wherein said remote computer combines said genetic risk factor and said environmental risk factor into a composite risk factor.

14. The system recited in claim 13 wherein said remote computer uses said composite risk factor to generate a prescribed drug therapy from a drug database.

15. The system recited in claim 14 wherein said remote computer transmits said prescribed therapy over said network to the personal computer originating the request.

16. The system recited in claim 10 wherein said testing centers have Laboratory Information Systems operating on a common data format.
